(19) United States
(12) Patent Application Publication
(10) Pub. No.: US 2018/0217086 A1
(43) Pub. Date: Aug. 2, 2018

(54) Mixed-Potential Electrochemical Sensor

(71) Applicants: Sandia Corporation, Albuquerque, NM (US); STC.UNM, Albuquerque, NM (US)

(72) Inventors: Fernando Henry Garzon, Santa Fe, NM (US); Lok-kun Tsui, Albuquerque, NM (US)

(21) Appl. No.: 15/423,067

(22) Filed: Feb. 2, 2017

(51) Int. Cl. G01N 27/407; G01N 33/00; G01N 27/30

(52) U.S. Cl. CPC G01N 27/4074; G01N 33/0037; G01N 27/30; G01N 33/0047; G01N 33/004

(57) ABSTRACT

A three-electrode mixed-potential electrochemical sensor coupled with an artificial neural network data analysis approach can extract concentrations from voltages and identify gas streams consisting of single and binary mixtures of NO₂, NO, CO, and C₃H₈. By using the data from the sensors in biased and unbiased mode, single and binary mixtures can be identified with >98% accuracy identify all single and binary mixtures. While concentrations can be readily extracted from single test gas mixtures through a linear fit to the most sensitive electrode pair, binary mixture concentrations analyzed with an artificial neural network resulted in error distributions with a 95% peak accuracy in concentration with 80% of the data points having an accuracy at the 88% level. The sensor is suitable for control and monitoring of diesel and gasoline engines, turbines, steam power plants, and other combustion technologies.

### STATEMENT OF GOVERNMENT INTEREST

This invention was made with Government support under contract no. DE-AC04-94AL85000 awarded by the U. S. Department of Energy to Sandia Corporation. The Government has certain rights in the invention.

### FIELD OF THE INVENTION

The present invention relates to chemical sensors and, in particular, to a mixed-potential electrochemical sensor that can be used to measure emissions, such as NOₓ, CO, NH₃, and hydrocarbons.

### BACKGROUND OF THE INVENTION

Lean burn gasoline and diesel engines use a high air-to-fuel ratio to ensure high fuel combustion efficiency and to lower CO and hydrocarbon (HC) emissions, but the excess oxygen partially reacts with nitrogen to create nitric oxides. Lean burn diesel engines therefore require a two-stage catalytic system to eliminate pollutants: an initial diesel oxidation catalyst is used to decompose CO and HCs and a urea-SCR system is used to reduce NOₓ to N₂ via an NH₃ mediated reaction. See M. V. Twigg, Appl. Catal. B Environ. 70, 2 (2007); J. Kašpar et al., Catal. Today 77, 419 (2003); and M. Koebel et al., Catal. Today 59, 335 (2000). Currently there are no sensors installed in automobiles that can quantitatively monitor the concentration of pollutants in the exhaust gas stream. Such sensors need to be robust in the atmosphere of exhaust gas, have low cost, and be able to distinguish between the different pollutant gases which may be present.

Mixed-potential electrochemical sensors are a promising technology for on board emissions monitoring in automobiles which meets these requirements. Mixed-potential electrochemical sensors comprise multiple dissimilar electrodes exposed to an analyte gas, typically a mixture containing oxygen and an oxidizable or reducible gas. Mixed potentials of different voltages develop on each electrode due to differences in electrokinetic redox rates of the dissimilar electrodes. The sensor response voltage is the difference in mixed potential attained by each electrode. For example, mixed potential sensors can take advantage of the difference in electrochemical kinetics of oxidation and reduction of a target pollutant gas and O₂ of two dissimilar electrodes embedded in a solid electrolyte. See J. W. Fergus, J. Solid State Electrochem. 15, 971 (2011); and F. H. Garzon et al., Solid State Ionics 136-137, 633 (2000). Sensors pairing Pt and La_{0.8}Sr_{0.2}CrO₃ (LSCO) have previously demonstrated high sensitivity to HCs at open circuit and NO under bias. See P. K. Sekhar et al., Sensors Actuators, B Chem. 144, 112 (2010); and P. K. Sekhar et al., Sensors Actuators B Chem. 183, 20 (2013). Pt and Au or Au alloys, such as Au/Pd, have proven to be sensitive electrode pairs for detection of both CO and NH₃. See P. K. Sekhar et al., Sensors Actuators, B Chem. 144, 112 (2010); C. R. Kreller et al., ECS Trans. 64, 105 (2014); and J. W. Fergus, Sensors Actuators, B Chem. 122, 683 (2007). The integration of these two types of sensing electrode pairs onto one device in principle would enable the detection of all species of interest to automotive emissions.

### SUMMARY OF THE INVENTION

The present invention is directed to a mixed-potential electrochemical gas sensing device. The robust sensor platform can measure EPA regulated emissions, such as NOₓ, CO, NH₃, and hydrocarbons, with high accuracy, sensitivity, and specificity. Data processing with Artificial Neural Networks (ANNs) provides >95% peak accuracy at discriminating individual components at the 50-250 PPM levels. The sensor enables real-time diagnostics, with response times less than 1/100 sec., and can operate in hostile high temperature combustion environments without the need for cooling or filtration. Therefore, the sensor can provide exhaust chemistry feedback information that can be used to improve combustion efficiency for diesel and gasoline engines, turbines, steam power plants, and other combustion applications. The sensor also enables the detection of explosive compounds with small handheld devices by providing a molecular fingerprint of the explosive compounds.

An exemplary device comprises Pt, La_{0.8}Sr_{0.2}CrO₃ (LSCO), and Au/Pd alloy electrodes and a porous yttria-stabilized zirconia (YSZ) electrolyte. The three-electrode design takes advantage of the preferential selectivity of the Pt+Au/Pd and Pt+LSCO pairs towards different species of gases and has additional tunable selectivity achieved by applying a current bias to the latter pair. As an example of the invention, voltages were recorded in single and binary gas streams of NO, NO₂, C₃H₈, and CO. ANNs were trained to examine the voltage output from sensors in biased and unbiased modes to both identify which single test gas or binary mixtures of two test gases were present in a gas stream as well as extract concentration values. With this technique, binary and single gas mixtures of NO, NO₂, CO, and C₃H₈ can be identified with >98% accuracy. The ANNs can also recover concentration values from voltages with peak error of 5%. Concentration values with peak error of 10% can be extracted from a ternary mixture of NO₂, CO, and C₃H₈.

### BRIEF DESCRIPTION OF THE DRAWINGS

The detailed description will refer to the following drawings, wherein like elements are referred to by like numbers.

FIG. 1 is a schematic illustration of a three-electrode sensor. The front side contains the Pt/LSCO/AuPd electrodes while the back side contains an integrated resistive heater.

FIGS. 2(a) and 2(b) are schematic illustrations of the architecture of artificial neural networks comprising a three-layer, fully-connected, feed forward structure with voltage as inputs and gas label or concentration values as outputs.

FIG. 2(a) is an illustration of a concentration network design. FIG. 2(b) is an illustration of a classification network design.

FIGS. 3(a)-(d) are graphs of voltage responses to single test gas samples of (a) NO₂, (b) NO, (c) CO, and (d) C₃H₈ for each of the sensor pairs in either biased LSCO/Pt mode or unbiased (UB) mode.

FIGS. 4(a)-(c) are graphs of visualization of decision boundaries for separating the gas mixtures using (a) only the unbiased sensor, (b) only using the biased sensor, (c) using both the unbiased and biased data. The innermost contour represents 80% confidence with decreasing steps of 20% confidence.

FIGS. 5(a)-(c) are confusion matrices for single and binary test gas classification task using data from (a) only unbiased sensor, (b) only the biased sensor, and (c) both the unbiased and unbiased data. All gas species can be accurately classified with >98% accuracy using both sensors.

FIGS. 6(a)-(f) are graphs of the error distribution of the concentration of binary mixtures of (a) NO₂+C₃H₈, (b) NO₂+CO, (c) NO₂+NO, (d) C₃H₈+CO, (e) C₃H₈+NO, and (f) CO+NO.

FIGS. 7(a)-(d) are histograms for the error in concentration as well as a cumulative fraction curve for ternary mixtures of CO, NO₂, and C₃H₈. (a) Total error based on vector difference, (b) Error for NO₂, (c) Error for CO, and (d) error for C₃H₈. NO₂ error is lower by a factor of 2 compared to CO and C₃H₈.

FIG. 8 is a graph of the average total error for ternary mixtures as a function of the number of data points used for training. Diminishing returns are reached after 50 training data points are used.

### DETAILED DESCRIPTION OF THE INVENTION

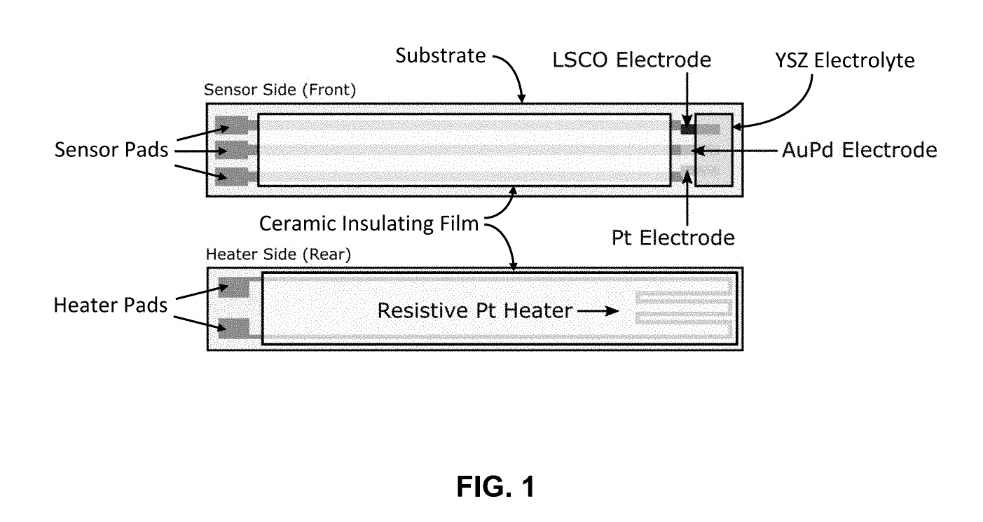

The present invention is directed to a mixed-potential electrochemical gas sensor. In FIG. 1 is shown an exemplary sensor that integrates dense Pt, LSCO, and Au_{0.5}Pd_{0.5} electrodes with a porous YSZ electrolyte. The selection of dense electrodes and a porous electrolyte is aimed at inhibiting the sintering of metal electrodes while the porous YSZ electrolyte is not expected to sinter within the operational temperature window of the sensor (400-600° C.). See P. K. Sekhar et al., Sensors Actuators, B Chem. 144, 112 (2010); C. R. Kreller et al., ECS Trans. 64, 105 (2014); and R. Mukundan et al., J. Electrochem. Soc. 150, H279 (2003). Accordingly, the substrate of this exemplary sensor was constructed by lamination of green insulator-YSZ-insulator tapes, onto which the electrodes and heater elements were deposited by screen printing and co-fired in the following order: heater and Pt leads fired at 1450° C., LSCO fired at 1200° C., Au/Pd fired at 1100° C., and the porous YSZ electrolyte fired at 1100° C. The integrated resistive Pt heater on the underside of the sensor can be used to set the temperature by applying a voltage across the heater terminals. Both the front and rear electrode leads are coated with a ceramic insulating film. Contacts to the sensors were made by Sn—Ag soldering Ag and Cu wires to the sensor and heater pads, respectively. Touch contacts can also be used. When the electrodes are exposed to an analyte gas, mixed potentials of different voltages develop on each electrode due to differences in electrokinetic redox rates of the electrodes. The sensor response voltage is the difference in mixed potential attained at each electrode.

For ease of collecting training data for biased and unbiased mode simultaneously, two three-electrode sensors were placed in 1″ glass tubes through which test gas was flown. The temperature of the sensors was set at 480° C. for NOₓ, C₃H₈, and CO detection by a Custom Sensors Solutions Rev H-0 board which adjusts the applied voltage with a feedback look to maintain a fixed resistance on the heater leads. A higher temperature of 530° C. is needed for NH₃ detection because the signal saturates. Gas mixing was controlled by an Environics 2000 gas mixer. Each sensor was exposed to a base gas of 10% O₂, 2.5% CO₂, and balance N₂ to simulate a dilute exhaust gas stream. NO₂, NO, C₃H₈, and CO were then injected at concentration levels of 75-250 ppm for C₃H₈ and 50-225 ppm for the others. For each pairwise combination of gases, data was collected at 25 ppm intervals, and an additional 30 randomly generated data points were obtained within each window. For ternary mixtures, data was collected between 50-150 ppm for CO and NO₂ and 75-175 ppm for C₃H₈ at 25 ppm intervals and 90 randomly generated data points were collected in this window. The total flow rate out of the gas mixer was 180 SCCM, nominally split across each sensor at 90 SCCM. Data was collected using two Keithley 2400 Sourcemeters, a Fluke 8842A digital multimeter, and three HP 34401A digital multimeters connected to each pair of electrodes in the polarity convention as follows: Au/Pd (+) and Pt(−), LSCO (+) and Pt (−), Au/Pd (+) and LSCO (−). One sensor was left at open circuit while the other sensor had a negative bias of −0.2 μA (−0.6 μA for ammonia) applied to the LSCO and Pt pairs by one of the Keithley 2400 Sourcemeters. For each data point, base gas was flowed for 10 minutes, the test gas was added for 10 minutes, and then the sensor was purged with base gas for 10 minutes.

### Artificial Neural Networks

The two unsolved challenges which remain after a dataset of voltage-concentration values have been collected are: identification of which pollutant gas species are present in a stream and what are their concentrations. Artificial neural networks (ANNs) can be used which are capable of learning relationships between voltage inputs and concentration or gas identity outputs without the need to specify their functional form ahead of time and which can be flexibly applied to both regression and classification tasks. See M. Kubat, An Introduction to Machine Learning, Springer, New York, N.Y. (2015). ANNs have already shown success in the sensor field, having been applied to chemical detection and monitoring of food quality. See K. J. Albert et al., Chem. Rev. 100, 2595 (2000); J. White et al., Anal. Chem. 68, 2191 (1996); A. Galdikas et al., Sensors Actuators, B Chem. 69, 258 (2000); and X. L. Wang et al., Proc. 2012 2nd Int. Conf. Instrum. Meas. Comput. Commun. Control. IMCCC 2012, 702 (2012).

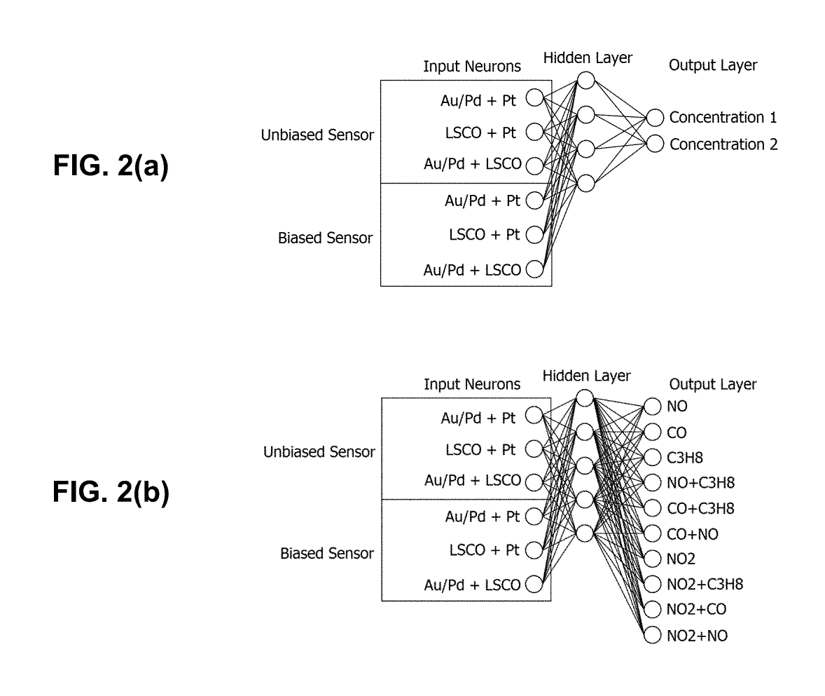

The ANNs used for data analysis with the present invention were structured in a way that can use data from the unbiased sensor, the biased sensor, or both, as shown in FIGS. 2(a) and 2(b). The ANN architecture consisted of 3 fully-connected layers with 4 hidden neurons in the concentration network design (FIG. 2(a)) and 5 hidden units in the classification network design (FIG. 2(b)). From each input neuron (Equation 1), the output of the input node is passed to the hidden layer. At the j-th hidden layer, the data from the i-th element in the input layer is multiplied by weights W_{2,i,j} and a bias b_{2,j} is added (Equation 2). A sigmoidal activation function is then applied before passing to the output layer (Equation 3). At the j-th output layer neuron, the data from the i-th hidden layer element are summed by weights W_{3,i,j} and a bias b_{3,j} is added. A sigmoidal activation function was used at the output layer for the concentration network (Equation 4) and the softmax function for the classification network (Equation 5). The bias terms and the weight terms are the fitting parameters that must be adjusted to minimize error.

$$\begin{matrix} {a}_{1 , j} = {x}_{i} & [ 1 ] \\ {a}_{2 , j} - \frac{1}{1 - exp ( {z}_{2 , j} )} ; {z}_{2 , j} - \sum {a}_{1 , i} {w}_{2 , i , j} + {b}_{2 , j} & [ 2 ] \\ {z}_{2 , j} ≡ \sum {a}_{2 , i} {W}_{3 , i , j} + {b}_{3 , j} & [ 3 ] \\ \text{?} = \frac{1}{1 - exp ( \text{?} )} & [ 4 ] \\ \text{?} = \frac{exp ( \text{?} )}{\sum exp ( \text{?} )} & [ 5 ] \\ E = \frac{1}{2 m} \sum {( {C}_{ANN} ( {W}_{2} , {b}_{2} , {W}_{3} , {b}_{3} , {x}_{i} ) - {C}_{sensor} )}^{2} \\ \text{?} \text{indicates text missing or illegible when filed} & [ 6 ]\end{matrix}$$

The error function to be minimized is the average reduced squared error for m training data points (Equation 6) where for each data point, C_{ANN} is the result from the ANN and Cₛₑₙₛₒᵣ is the result from the experimental data. The ANN was implemented using the PyBrain (version 0.3.3) and NumPy (version 1.10.1) packages for the Python programming language (Python 2.7). See T. Schaul et al., J. Mach. Learn. Res. 11, 743 (2010). Other commercial neural network/machine learning toolkits can also be used. The data points were normalized to 250 ppm for the concentration values and 100 mV for the voltage values prior to input into the neural network. Each single gas or binary mixture was identified for classification tasks as a vector of 10 elements as seen in the output layer of the classification ANN in FIG. 2(b) with a 1 in the index of the correct identification and a 0 elsewhere. A gradient descent algorithm was used with the gradient calculated by back propagation for minimization of the error. See R. D. Reed and R. J. Marks, Neural Smithing: Supervised Learning in Feedforward Artificial Neural Networks, MIT Press, Cambridge, Mass., (1999). The datasets of all single and binary gas mixtures were collected and split into 20% test and 80% training subsets at random to gauge the accuracy of the neural network at gas mixture identification. Then the ANN was trained for 400 iterations on the training subset only, and accuracy was calculated based on the test dataset. For the accuracy of concentrations in binary and ternary mixtures, datasets from each binary mixture were split into 20% test and 80% training subsets at random and 10,000 iterations of training were used to assess the accuracy of the neural networks. This random sampling was repeated 30 times for the classification task and 30 times for reading concentrations from each binary mixture using data from only the unbiased sensor, only the biased sensor, and both sensors.

### Data Visualization Via Principle Component Analysis

Since it is impossible to draw 6-dimensional data and difficult to represent 3-dimensional data on a 2D graphic, principle component analysis (PCA) was used to compress the higher dimensional data down to 2D. PCA calculates the 2D plane in the higher dimensional space which minimizes the projection distance between data points and the plane. See E. Alpaydin, Introduction to Machine Learning, The MIT Press, Cambridge, Mass., (2004). With m data points and n voltage measurements per data point, the m×n matrix X is constructed:

$${X}_{n} = [ \begin{matrix}{v}_{0}^{0} & {v}_{1}^{0} & {v}_{2}^{0} & {v}_{3}^{0} & … & {v}_{n}^{0} \\ {v}_{0}^{1} & {v}_{1}^{1} & {v}_{2}^{1} & {v}_{3}^{1} & … & {v}_{n}^{1} \\ {v}_{0}^{2} & {v}_{1}^{2} & {v}_{2}^{2} & {v}_{3}^{2} & … & {v}_{n}^{2} \\ … & … & … & … & … & … \\ {v}_{0}^{m} & {v}_{1}^{m} & {v}_{2}^{m} & {v}_{3}^{m} & … & {v}_{n}^{m}\end{matrix} ] \qquad [ 7 ]$$

Let the covariance matrix

$$C ≡ \frac{1}{m} {X}_{n}^{T} * {X}_{n}$$

where X^{T} represents the matrix transpose operation of a matrix X and * represents matrix multiplication. The singular value decomposition (SVD) of C produces three matrices in Equation 8.

U,S,V=SVD(C) [8]

To reduce the dimensionality down from higher dimension n to 2 dimensions, the first 2 columns of the matrix U are computed from SVD, defined as U₂ and multiply it by Xₙ. Here, U₂ is an n by 2 matrix, Xₙ is an m by n matrix, and X₂ is an m by 2 matrix (Equation 9). The diagonal entries of the S matrix, the singular values, can be used to determine how many linearly independent parameters are present. In this case, since the Au/Pd+LSCO electrode pair measures the difference between the Au/Pd+Pt and LSCO+Pt electrodes, 4 non-zero singular values were found and 4 linearly independent measurements per data point were collected.

X₂=Xₙ*U₂ [9]

See M. T. Heath, Scientific Computing, McGraw-Hill, New York, N.Y., 2002. It is also possible to approximate n-dimensional data from a k-dimensional value. From a vector zₖ of dimension k, the approximation of the vector in n-dimensions zₙ is given by:

zₙ=Uₖ*zₖ [10]

This can be used to visualize decision boundaries for classification where the dataset from either the 3-dimensional voltage space of a single sensor or 6-dimensional voltage space of both unbiased and biased sensors is projected into 2D PCA space. Then, data points can be sampled within the window of 2D PCA space and projected back up into 3- or 6-dimensional voltage space using Equation 10. A contour plot of the confidence of the network is generated at the sampled position in 2D PCA space, defined as the ratio of the output node of a gas mixture label with the highest value to the sum of the output nodes in the classification ANN at the sampled positions.

### Voltage Response to Single Test Gases

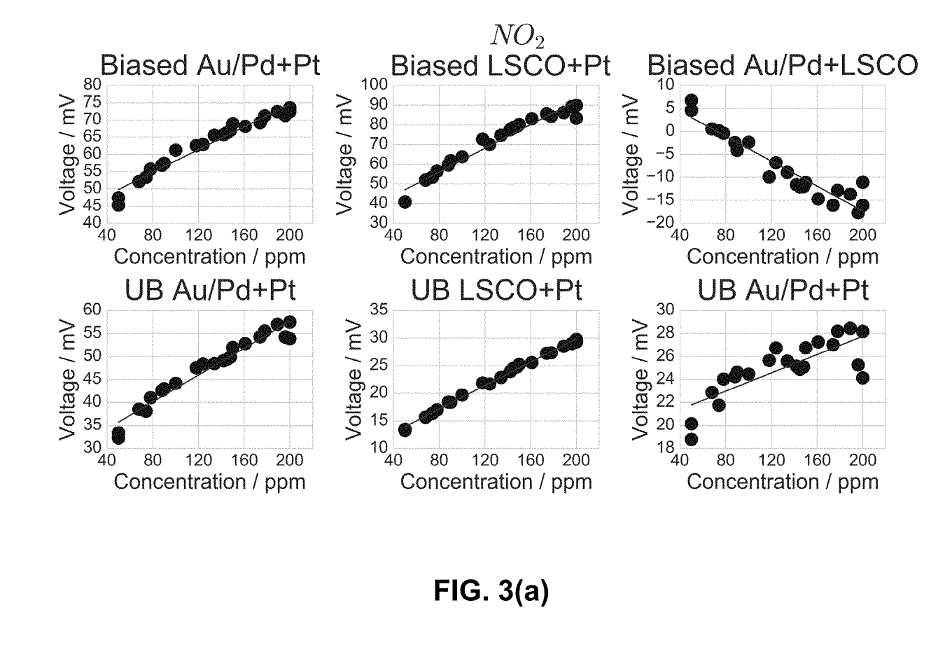

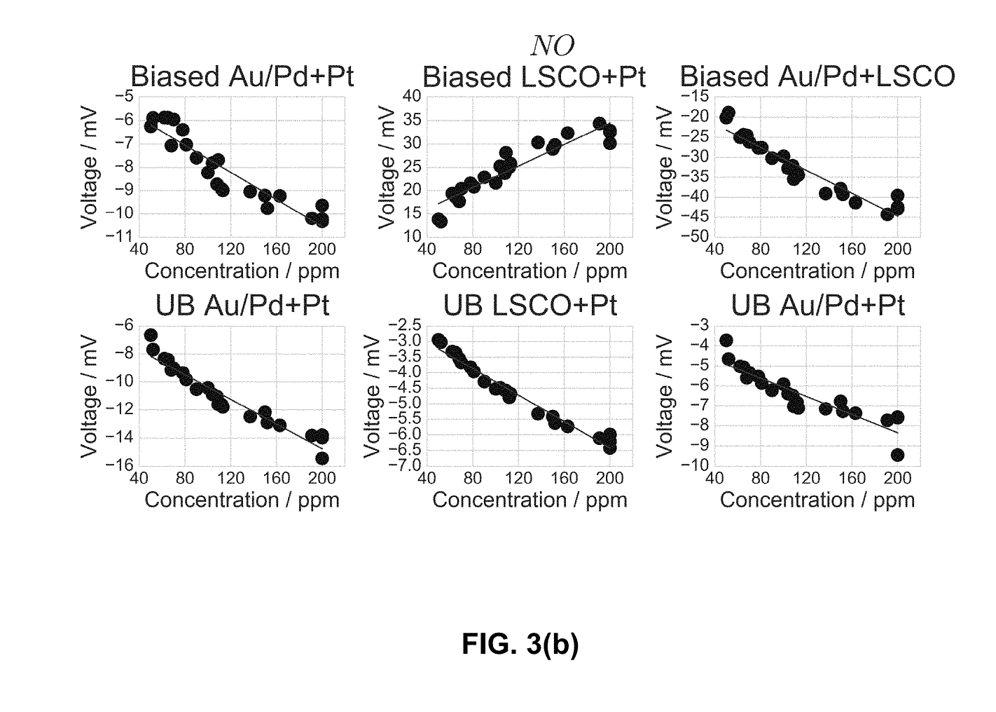

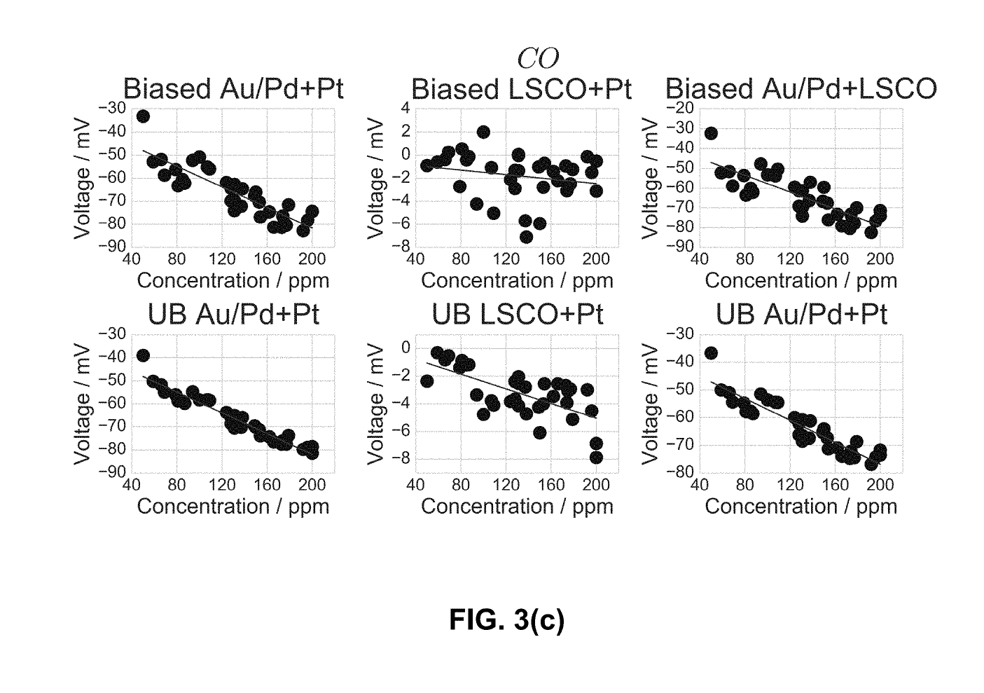

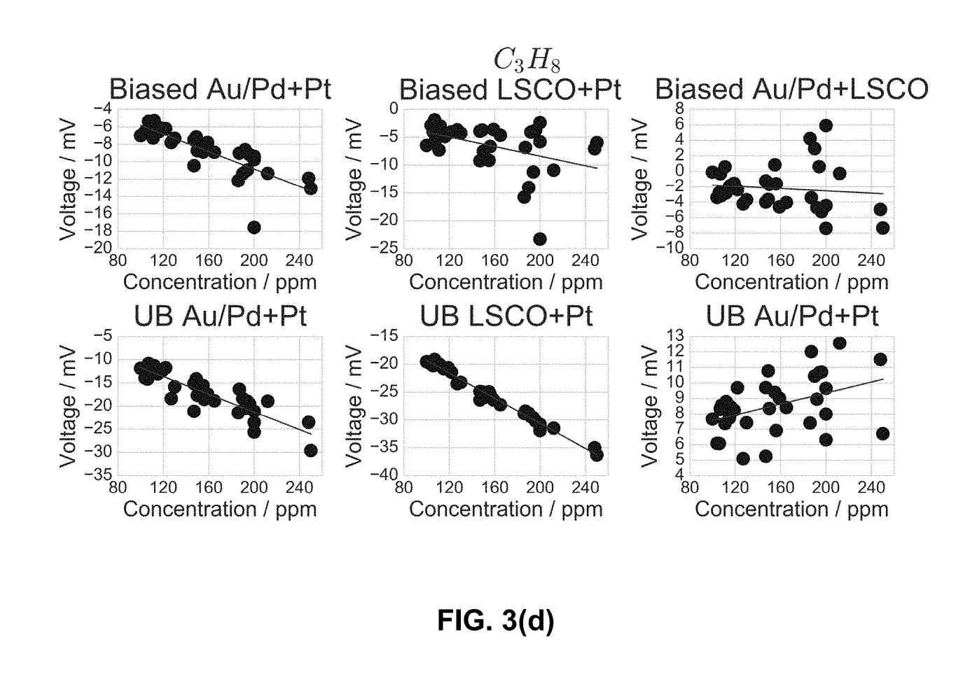

Prior to analyzing binary gas responses, the response of the three-electrode sensor in biased and unbiased mode was tested against varying concentrations of single test gases, as shown in FIGS. 3(a)-(d). The responses to single gases within a 50-250 ppm window are linear enough that a straight line can be drawn to fit the sensor response to back out a concentration-voltage relation. Based on this data, how the sensitivity changes with materials selection and current bias can be used to see which electrode performs best as a sensor for a particular test gas species. The quantitative criteria used are the difference in the voltage between the minimum and maximum concentrations, representing a sensor pair with a high range of sensitivity, and goodness of fit parameter R² for a linear fit through the data, as shown in Table. The sensor's LSCO+Pt and Au/Pd+LSCO pairs in biased mode excel at detecting NOx species, while in open circuit mode the sensor best suited for detecting CO and C₃H₈ uses the Au/Pd+Pt and LSCO+Pt pairs, respectively. These results are consistent with the previously discussed studies that established which of the LSCO or Au electrodes paired with Pt would be effective sensing electrodes for the four species in this experiment. See P. K. Sekhar et al., Sensors Actuators, B Chem. 144, 112 (2010); and P. K. Sekhar et al., Sensors Actuators B Chem. 183, 20 (2013).

TABLE 1
For each single test gas mixture, the ΔV value is the magnitude of the difference between
the voltage at the highest and lowest concentration and Linfit R² value is the goodness
of fit parameter for a linear fit of concentration vs. voltage through the data. The cell
with an asterisk represents the electrode pair with the best of both parameters.

| Test | | Biased Sensor | | | Unbiased Sensor | | |
|---|---|---|---|---|---|---|---|
| Gas | Data | Au/Pd + Pt | LSCO + Pt | Au/Pd + LSCO | Au/Pd + Pt | LSCO + Pt | Au/Pd + LSCO |
| C₃H₈ | ΔV | 12 mV | 21 mV | 13 mV | 18 mV | 17 mV* | 7 mV |
| | Linfit | 0.66 | 0.18 | 0.01 | 0.78 | 0.98 | 0.17 |
| | R² | | | | | | |
| CO | ΔV | 50 mV | 9 mV | 50 mV | 42 mV* | 8 mV | 40 mV |
| | Linfit | 0.75 | 0.05 | 0.68 | 0.93 | 0.44 | 0.85 |
| | R² | | | | | | |
| NO | ΔV | 4 mV | 21 mV | 25 mV* | 9 mV | 3 mV | 6 mV |
| | Linfit | 0.88 | 0.87 | 0.91 | 0.93 | 0.97 | 0.82 |
| | R² | | | | | | |
| NO₂ | ΔV | 28 mV | 49.11* | 24 mV | 25 mV | 17 mV | 10 mV |
| | Linfit | 0.95 | 0.94 | 0.90 | 0.95 | 0.99 | 0.63 |
| | R² | | | | | | |

While a linear fit is suitable for single gas mixtures, the voltage response when binary and ternary mixtures are present is more complex due to the interactions among the different gases. Examining the voltage response on the Au/Pd+Pt pair at open circuit, a voltage response of −14 mV is associated with 200 ppm of NO, −80 mV is associated with 200 ppm of CO, and −40 mV is seen with 200 ppm of NO and 200 ppm of CO. The responses of binary mixtures cannot be strictly additive because of reactions such as that in Equation 11, in which CO may act as a reducing agent to produce inert products CO₂ and N₂.

2CO+2NO→2CO₂+N₂ [11]

Accounting for these possible reactions in the gas stream without needing to explicitly define a functional form for these cross interference effects necessitates a flexible analysis technique that artificial neural networks are well suited for.

### Classification Accuracy

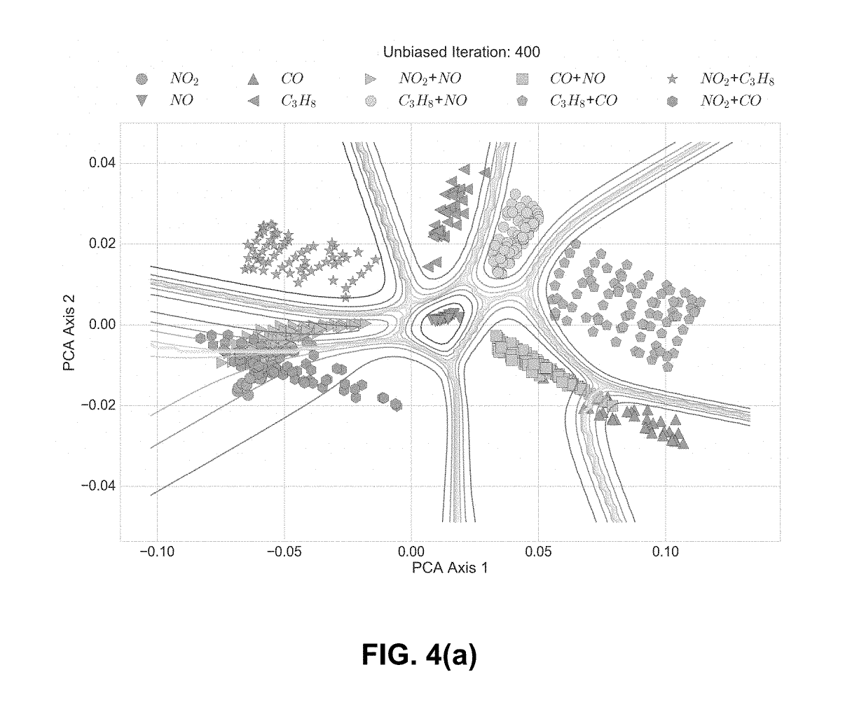

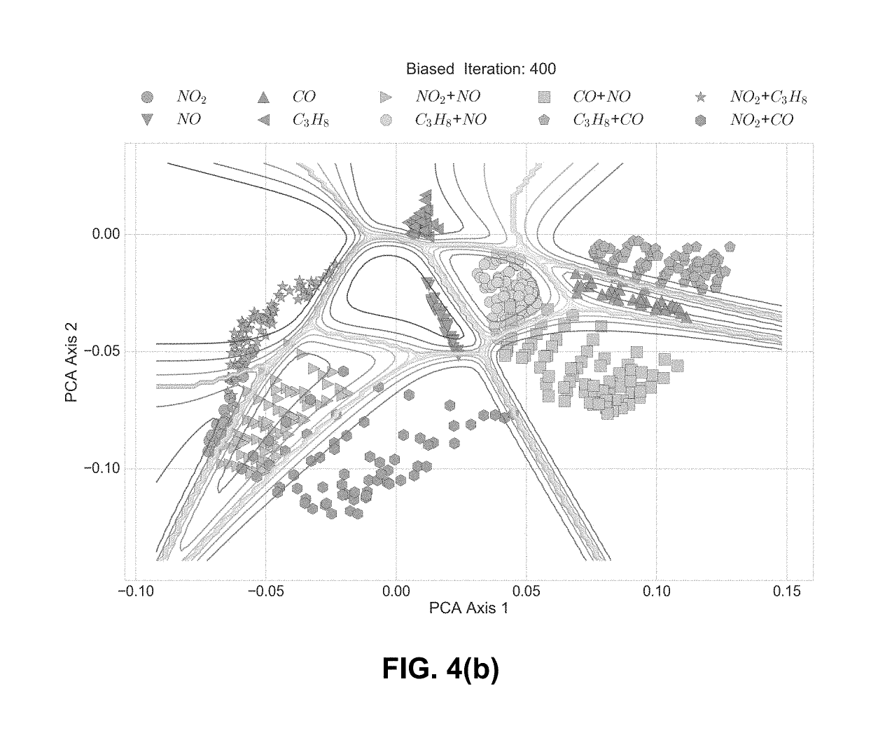

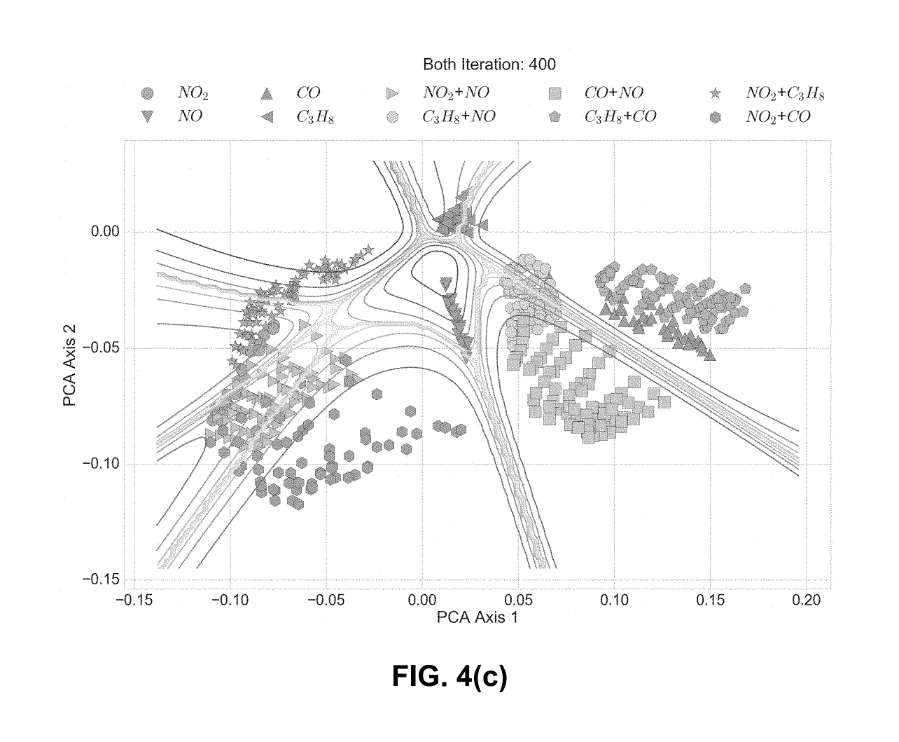

The distribution of voltage signals generated by the sensors when exposed to the different gas mixtures in 2D PCA space is shown in FIG. 4(a)-(c) for the unbiased sensor, the biased sensor, and both sensors, respectively. As shown in FIG. 4(a), the unbiased sensor has overlaps between the NO₂+CO, the NO₂+NO, and the NO₂-only signals in the lower left quadrant as well as the CO+NO and the CO-only signals in the lower right quadrant. Consequently, the boundaries in the ANN confidence between these mixtures are poorly defined. Because of the increased sensitivity of the biased sensor under current bias to NOₓ, the overlapping NO containing species are spread out over a wider area of the lower left quadrant of FIG. 4(b), which facilitates separation of these signals. Additionally, the CO+NO and the CO-only signals have become well separated. As shown in FIG. 4(c), little difference in the position in 2D PCA space is seen in the distribution when both sensors are used—any advantages in separation capability for using both sensors must be taking advantage of higher dimensional data.

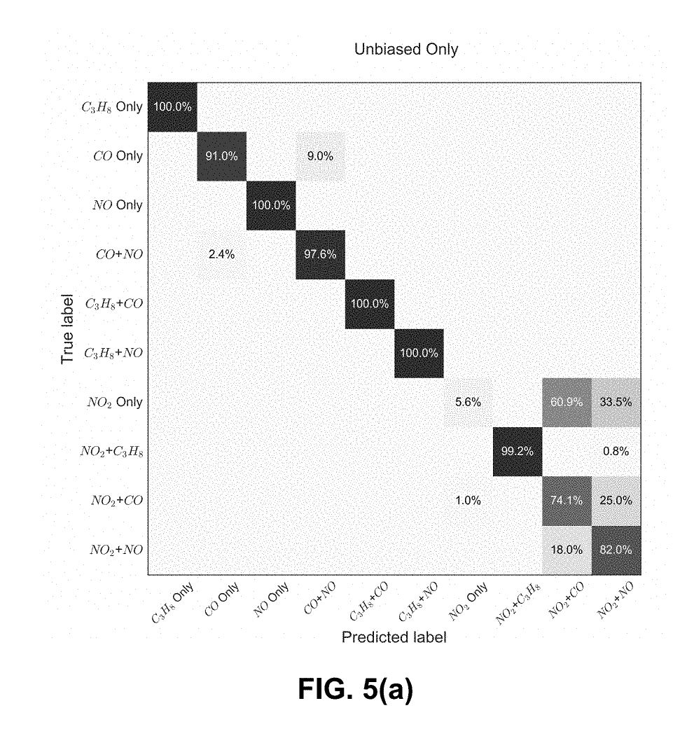

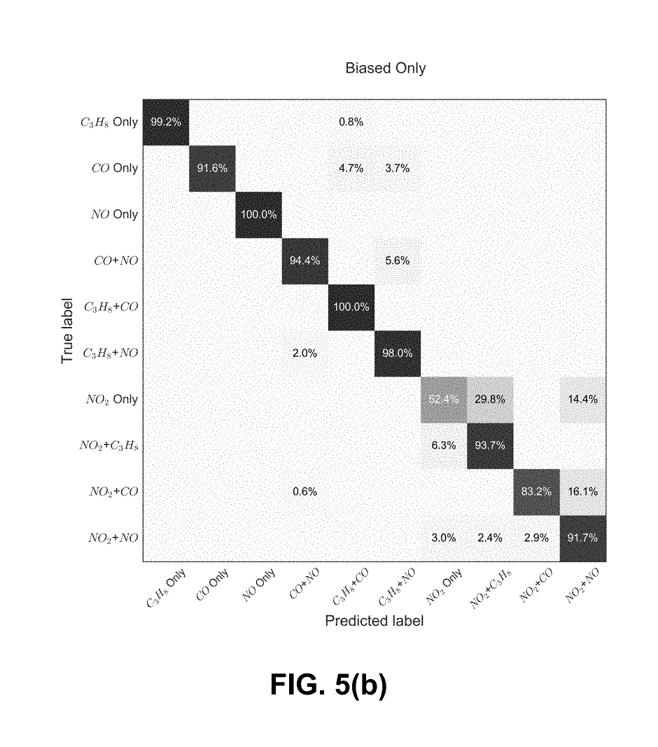

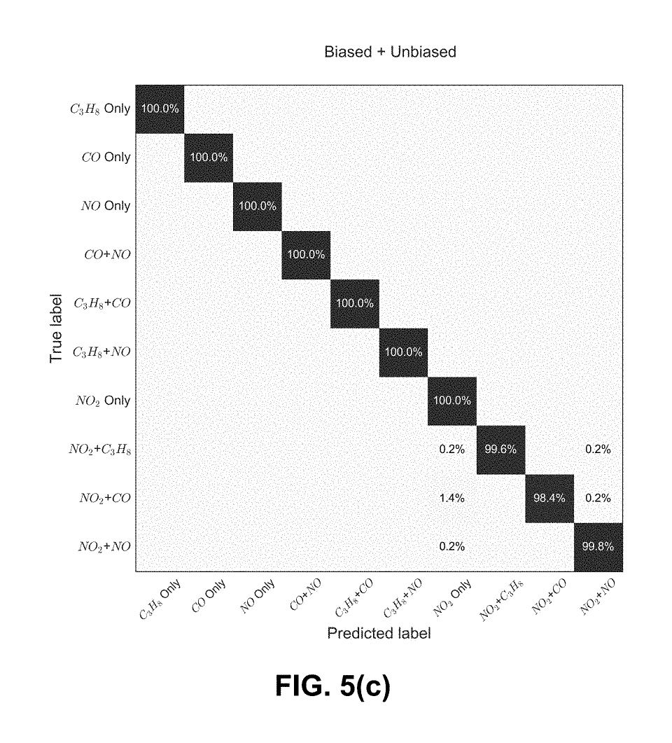

Confusion matrices are plotted in FIG. 5(a)-(c) to assess the classification performance quantitatively. The figures show the true label on the vertical axis and the predicted label on the horizontal axis. Values along the diagonal represent a classification of signals from a test gas stream into a correct label, and those off diagonal represent a misclassification. As shown in FIG. 5(a), networks trained on only the data from the sensor in unbiased mode perform poorly on the labels NO₂-only, NO₂+CO, and NO₂+NO with accuracies of 5.6%, 74.1%, and 82.0% respectively. If the networks are trained only on the biased data, as shown in FIG. 5(b), the accuracy for these three problematic gas species improves to 52.3%, 83.2%, and 91.7% due to the better separation for these labels in voltage space. Finally, taking advantage of separation in higher dimensions, FIG. 5(c) shows that every combination of single and binary test gas stream studied in this experiment can be classified with at least 98% accuracy.

### Concentration Accuracy

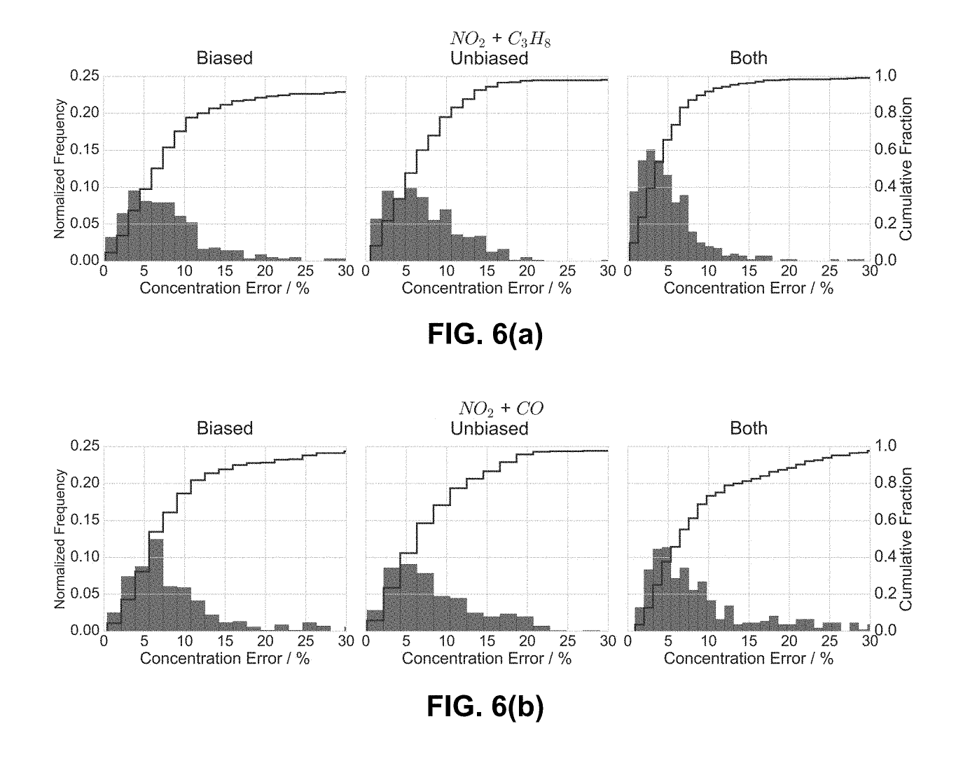

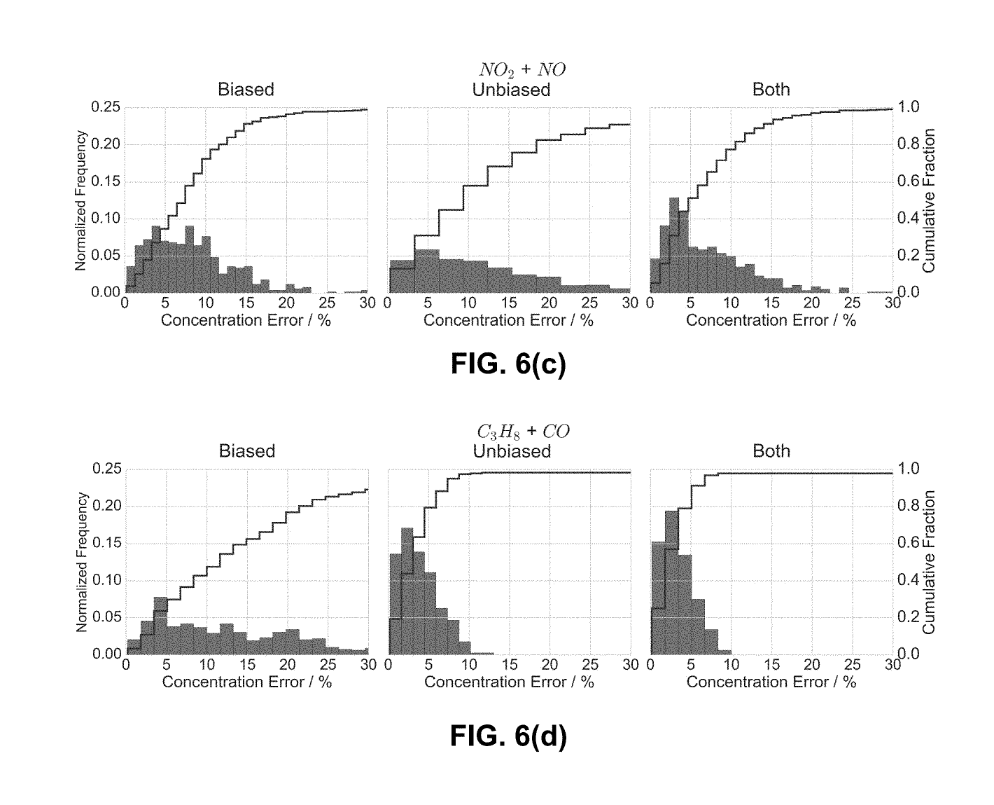

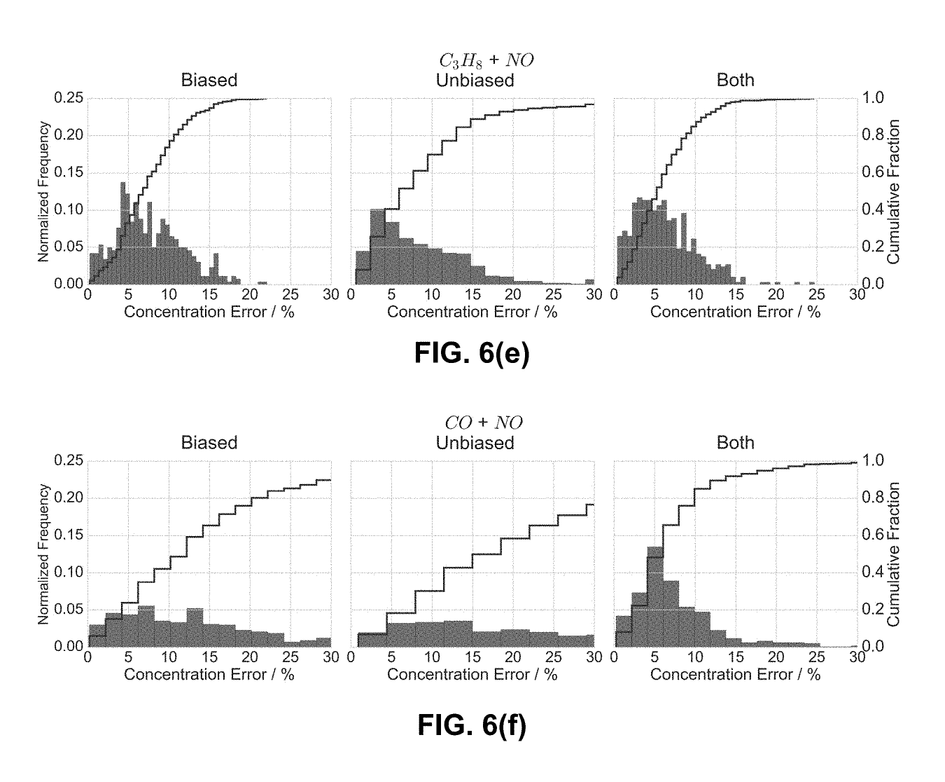

FIGS. 6(a)-(f) show the concentration accuracy of tests on neural networks trained on only the sensor in biased mode, only the sensor in unbiased mode, and both biased and unbiased sensor data. For each dataset approximately 60 data points were used in the training subset and 15 data points were used in the test subset. The error is defined as the norm of the vector difference between the real and predicted concentrations, normalized to the real concentrations (Equation 12). These distributions contain the error from all the test data points from 30 iterations of splitting each binary mixture dataset into test-train subsets.

$${E}_{c} = \frac{‖ {C}_{real} - {C}_{predicted} ‖}{‖ {C}_{real} ‖} \times 100 \% \qquad [ 12 ]$$

The best result combining features from both sensors was from C₃H₈+CO where the peak error was 2.5% and >95% of test data is confined to less than 10% error, as seen in FIG. 6(d). The most difficult pair for the networks to get concentration values from was the NO₂+CO pair which has a long tail extending out beyond 15-25%, as seen in FIG. 6(b). The typical peak in the error distribution for all other binary mixtures is 2-5% and 80% of test data points result in less than 12% error. The worst errors for the ANNs trained on the data from the biased sensor only are seen in the C₃H₈+CO mixture, as seen in FIG. 6(d). NO₂+NO and CO+NO are less accurate if only data from the unbiased sensor is used, as seen in FIGS. 6(c) and 6(f). In the case of the biased sensor, poor accuracy to C₃H₈+CO is expected since applying a bias is intended to suppress the signal from these two gases on the LSCO+Pt electrode in favor of NOₓ. It was shown earlier in Table 1 that the unbiased sensor has low sensitivity to NOx species. From these observations, the ANNs are capable of synthesizing the data from both biased and unbiased sensors and taking advantage of the best sensitivity of the two sensor modes.

### Ternary Gas Mixture Concentration Accuracy

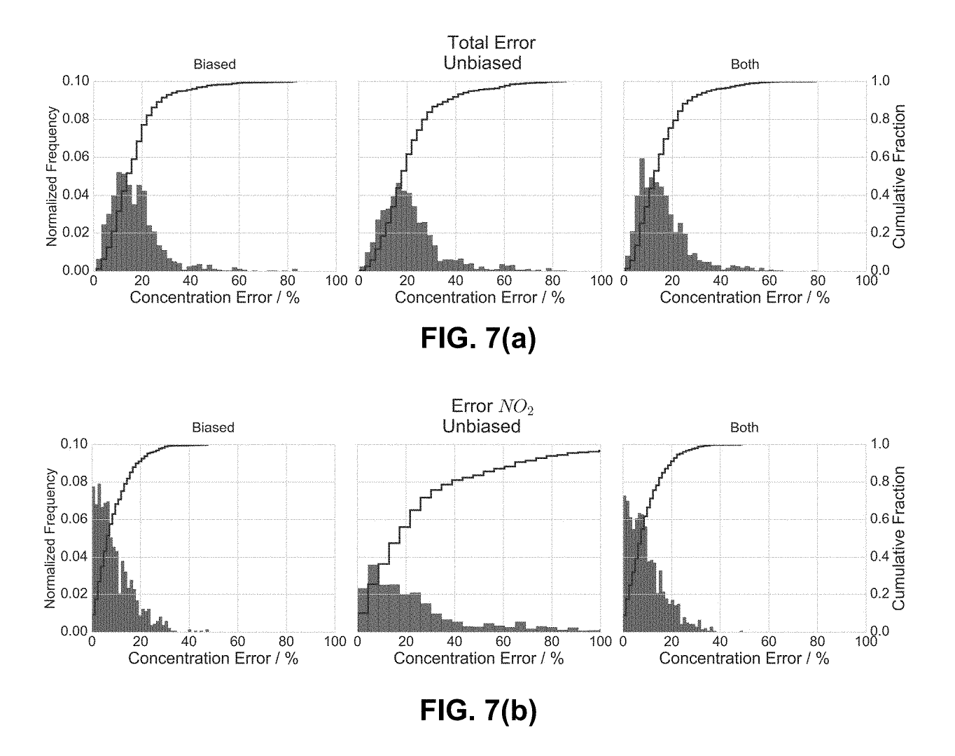

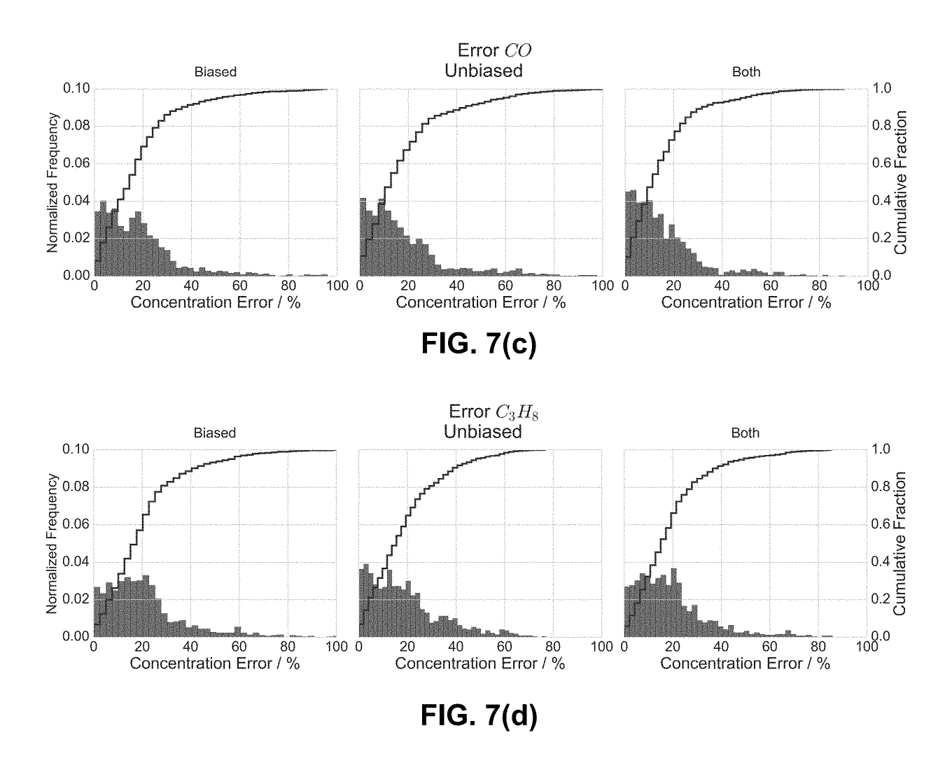

The final test for the artificial neural network was to extract concentration values from a ternary mixture of NO₂, C₃H₈, and CO. Due to mass flow controller limitations on the Environics system, values were restricted to a range of concentrations between 75-175 ppm for C₃H₈ and 50-150 ppm for NO₂ and CO for a total of 148 training data points and 37 test data points for each split. The artificial neural network was modified only by adding an additional neuron to the output layer. The test error distribution is plotted in FIG. 7(a) for a total vector difference error as used for binary mixtures and the error for the individual gases is broken down in FIGS. 7(b)-(d). The error in NO₂ is the smallest, coming from the better sensitivity of the sensor in biased mode. The mixture of NO₂+CO was found to have the largest error among the binary mixture concentration tests above, indicating that cross interference of this mixture will result in large errors in a ternary mixtures using the same number of inputs. The total concentration peaks at 10% and has 80% of the data bounded by 20% error, approximately a 2× increase over the binary mixtures. For ternary mixtures, the number of data points to be collected scales with the cube of the concentration window size to be sampled, so it is important to understand how many data points can be collected before accuracy no longer improves with more data.

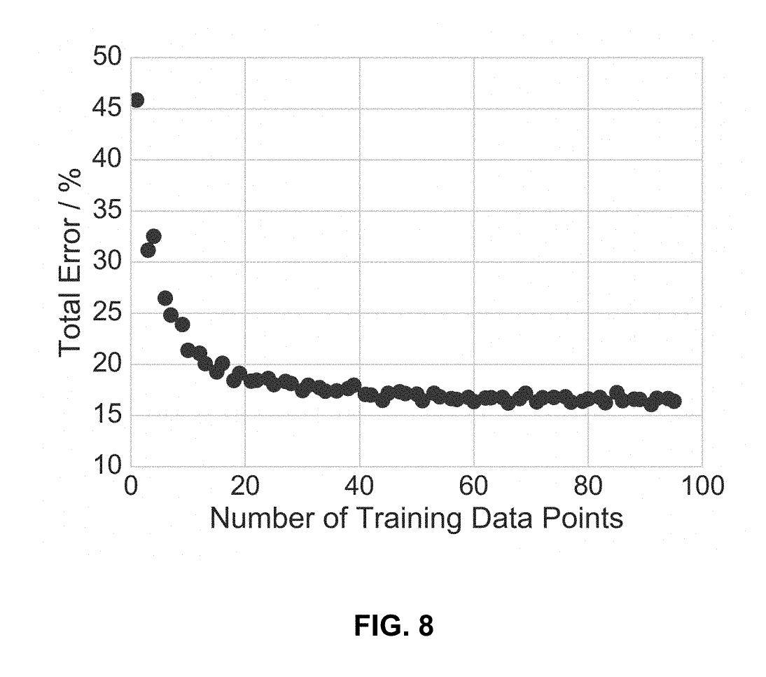

FIG. 8 shows the average total error for 37 test data points averaged over 30 random test-train splits while varying the number of training data points from 1 to 95. Beyond 50 data points there are diminishing returns on adding additional data points; collecting additional data points in this 100 ppm×100 ppm×100 ppm concentration window will not improve the accuracy of concentration predictions further. Options to enhance the accuracy can come from the acquisition of additional features, such as collecting sensor readings at different temperatures or using multiple current biases in addition to open circuit and a single applied bias.

The present invention has been described as a mixed-potential electrochemical sensor. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A mixed-potential electrochemical sensor, comprising:
a substrate,
at least three dissimilar electrodes disposed on the substrate with a porous electrolyte disposed therebetween, thereby forming at least three two-electrode pairs, wherein each of the two-electrode pairs has a preferential selectivity towards different species in a gas mixture exposed to the porous electrolyte,
an integrated resistive heater to heat the porous electrolyte to an operating temperature,
a voltage meter for measuring the mixed potentials developed on each of the two-electrode pairs due to differences in electrokinetic redox rates of the different species at each electrode, and
an artificial neural network processor to analyze the different species and their concentration in the gas mixture from the mixed potentials measured on each of the two-electrode pairs.

2. The sensor of claim 1, wherein the at least three dissimilar electrodes comprise Pt, LSCO, and Au/Pd.

3. The sensor of claim 1, wherein the porous electrolyte comprises porous yttria-stabilized zirconia.

4. The sensor of claim 1, wherein the different species comprise NO, NO₂, C₃H₈, or CO.

5. The sensor of claim 1, wherein the different species comprise hydrocarbons.

6. The sensor of claim 5, wherein the different species comprise parafins, olefins, and aromatics.

7. The sensor of claim 1, wherein the operating temperature is between 400° C. and 600° C.

8. The sensor of claim 1, wherein each of the two-electrode pairs is unbiased.

9. The sensor of claim 1, wherein a bias current is applied to at least one of the two-electrode pairs.
